(19) United States
(12) Patent Application Publication
(10) Pub. No.: US 2008/0139929 A1
(43) Pub. Date: Jun. 12, 2008

(54) SYSTEM AND METHOD FOR TRACKING AN INVASIVE SURGICAL INSTRUMENT WHILE IMAGING A PATIENT

(75) Inventors: John H. McGibbon, Boxborough, MA (US); Bruce Allen Cormier, Methuen, MA (US); Peter Traneus Anderson, Andover, WI (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/567,445

(22) Filed: Dec. 6, 2006

(51) Int. Cl. A61B 5/05

(52) U.S. Cl. 600/424

(57) ABSTRACT

A system and method for tracking an invasive surgical instrument provide time gaps between transmissions and receptions of electromagnetic energy, during which an imaging system, for example, and x-ray imaging system, can capture and update a display image. In some arrangements, data collection periods can be interleaved.

### CROSS REFERENCE TO RELATED APPLICATIONS

Not Applicable.

### STATEMENT REGARDING FEDERALLY SPONSORED RESEARCH

Not Applicable.

### FIELD OF THE INVENTION

This invention relates generally to surgical systems and methods and, more particularly, to a system and method that can track an invasive surgical instrument generally at the same time that another image, for example, an x-ray image, is being captured and displayed.

### BACKGROUND OF THE INVENTION

Tracking (or navigation) systems that can track the position of a surgical instrument within the body during a medical procedure are known. The tracking systems employ various combinations of transmitting antennas and receiving antennas adapted to transmit and receive electromagnetic energy. Some types of conventional tracking system are described in U.S. patent application Ser. No. 10/611,112, filed Jul. 1, 2003, entitled “Electromagnetic Tracking System Method Using Single-Coil Transmitter,” U.S. Pat. No. 7,015,859, issued Mar. 21, 2006, entitled “Electromagnetic Tracking System and Method Using a Three-Coil Wireless Transmitter,” U.S. Pat. No. 5,377,678, issued Jan. 3, 1995, entitled “Tracking System to Follow the Position and Orientation of a Device with Radiofrequency Fields,” U.S. Pat. No. 5,251,635, issued Oct. 12, 1993, entitled “Stereoscopic X-Ray Fluoroscopy System Using Radiofrequency Fields,” U.S. Pat. No. 6,980,921, issued Dec. 27, 2005, entitled “Magnetic Tracking System,” and U.S. Pat. No. 6,774,624, issued Aug. 10, 2004, entitled “Magnetic Tracking System.”

Some tracking systems have been adapted to track flexible probes inserted into the body for minimally-invasive surgeries, for example, nasal surgeries. One such system is described in U.S. Pat. No. 6,445,943, issued Sep. 3, 2002, entitled “Position Tracking System for Use in Medical Applications.” Each of the aforementioned patent applications and patents are incorporated by reference herein in the entirety.

The above-mentioned systems generally use one or more antennas positioned on a surgical instrument, which transmit electromagnetic energy, and one or more antennas positioned near a patient to receive the electromagnetic energy. Computational techniques can resolve the position, and in some systems, the orientation, of the surgical instrument. The systems are generally reciprocal, so that the transmitting antennas can be interchanged with the receiving antennas.

Imaging systems, for example, x-ray fluoroscopy systems and computer-aided tomography (CT) systems, can also track a surgical instrument within the body. Conventional x-ray fluoroscopes and CT systems are designed to minimize X-ray exposure. Nevertheless, the accumulated x-ray exposure to the patient can become significant, particularly during long procedures.

The above-described tracking systems mitigate the exposure of patients and staff to ionizing radiation, such as x-ray radiation, by providing an ability to track the surgical instrument using non-ionizing electromagnetic energy.

Though the tracking systems have mitigated exposure to ionizing radiation, nevertheless, sometimes it is still desirable during a surgical procedure in which an electromagnetic tracking system is utilized, to image a patient with an imaging system, e.g., a x-ray fluoroscopy system or a CT system, during a surgical procedure, once or from time to time during the procedure.

It is know that electromagnetic energy emitted by and used by a tracking system tends to cause a degradation of images generated by other systems, in particular, systems that use flat panel x-ray detectors (FPDs). Therefore, the other imaging system cannot operate effectively at the same time as the tracking system and still provide good images. It is not desirable to turn off the tracking system during the imaging by the other system, since during that time, the tracking provided by the tracking system would be unavailable. It is desirable to maintain a rapid update rate (i.e., frame rate) with the tracking system, without pauses.

### SUMMARY OF THE INVENTION

In accordance one aspect of the present invention, a method of processing a signal to track a surgical instrument includes transmitting, in a plurality of time windows separated from each other in time, a respective plurality of electromagnetic signals having a respective plurality of time distributions. The plurality of electromagnetic signals includes a first electromagnetic signal and a second electromagnetic signal. The first electromagnetic signal is transmitted during a first time window of the plurality of time windows, and has one or more narrowband frequencies. The first electromagnetic signal has a first time distribution in the first time window. The second electromagnetic signal is transmitted during a second time window of the plurality of time windows, and has the one or more narrowband frequencies. The second electromagnetic signal has a second time distribution in the second time window. The first and second time windows are each shorter than a collection period. The second time window is separated in time by a time gap from the first time window, which is sufficiently long to allow an image to be generated during the time gap.

The method further includes receiving the first electromagnetic signal during the first time window, receiving the second electromagnetic signal during the second time window, processing the first electromagnetic signal together with at least the second electromagnetic signal to provide a magnitude of the one or more narrowband frequencies, and processing the magnitude of the one or more narrowband frequencies in order to track a position of the surgical instrument.

In accordance with another aspect of the present invention, apparatus for processing a signal to track a surgical instrument includes a transmitter adapted to transmit, in a plurality of time windows separated from each other in time, a respective plurality of electromagnetic signals having a respective plurality of time distributions. The plurality of electromagnetic signals includes a first electromagnetic signal and a second electromagnetic signal. The first electromagnetic signal is transmitted during a first time window of the plurality of time windows and has one or more narrowband frequencies. The first electromagnetic signal has a first time distribution in the first time window. The second electromagnetic signal is transmitted during a second time window of the plurality of time windows and has the one or more narrowband frequencies. The second electromagnetic signal has a second time distribution in the second time window. The first and second time windows are each shorter than a collection period. The second time window is separated in time by a time gap from the first time window, which is sufficiently long to allow an image to be generated during the time gap. The apparatus also includes a receiver adapted to receive the first electromagnetic signal during the first time window and the second electromagnetic signal during the second time window, at least one magnitude processor adapted to process the first electromagnetic signal together with at least the second electromagnetic signal to provide a magnitude of the one or more narrowband frequencies, and a position and orientation generator adapted to process the magnitude of the one or more narrowband frequencies in order to track a position of the surgical instrument.

In accordance with another aspect of the present invention, a method of processing a signal to track a surgical instrument, includes collecting and processing electromagnetic signals in first and second collection periods, wherein the first and second collection periods are interleaved.

In accordance with another aspect of the present invention, apparatus for processing a signal to track a surgical instrument includes a processor adapted to collect and to process electromagnetic signals in first and second collection periods, wherein the first and second collection periods are interleaved.

With the above arrangements, it is possible to track a surgical instrument with a tracking system while also capturing images, e.g., x-ray images, of the patient with an imaging system. A so-called “tracking image” is representative of a position of a surgical instrument (i.e., position data) superimposed on or otherwise combined with an image of the patient. However the patient, or patient organs, e.g., the heart or lungs, can move during a surgical procedure. Movement of the patient or organs of the patient between the acquisition of the position data and the acquisition of images of the patient, which are combined to generate the tracking image, can reduce the accuracy of the resulting tracking image during the surgical procedure. Therefore, it is desirable to acquire with the tracking system a position of a surgical instrument as close as possible in time to the acquisition of the images with the imaging system.

With the above arrangements, it is also possible to collect a plurality of images of the patient and corresponding tracker position data while an x-ray arm is moving around the patient. The x-ray images can be processed to achieve three-dimensional images of the body and corresponding three-dimensional tracking images during the surgical procedure.

### BRIEF DESCRIPTION OF THE DRAWINGS

The foregoing features of the invention, as well as the invention itself may be more fully understood from the following detailed description of the drawings, in which:

FIG. 1 is a block diagram showing a system timing and control module that can control the operation of a transmitter module, a receiver module, and a position and orientation (P&O) module;

FIG. 1A is a block diagram showing further details of the receiver module of FIG. 1, having a plurality of magnitude processors; each magnitude processor having a respective vector processor;

FIG. 1B is a block diagram showing further details of one of the vector processors of FIG. 1A;

FIG. 2 is a set of graphs showing processing associated with a tracking system;

FIG. 3 is a set of graphs showing processing associated with another tracking system;

FIG. 4 is a set of graphs showing exemplary processing according to one embodiment of the present invention; and

FIG. 5 is a set of graphs showing exemplary processing according to another embodiment of the present invention, having interleaved processing.

### DETAILED DESCRIPTION OF THE INVENTION

Before describing the present invention, some introductory concepts and terminology are explained. As used herein, the term “quadrature” is used to describe a relationship between two signals, which have a phase relationship of approximately ninety degrees. In particular, a signal, when multiplied by a sine signal having a predetermined frequency, is in quadrature with the signal, when multiplied by a cosine signal having the same predetermined frequency. As is known, a sine signal and a cosine signal at the same frequency are separated in phase by ninety degrees, and are in quadrature. Therefore, the above-describe products are also in quadrature.

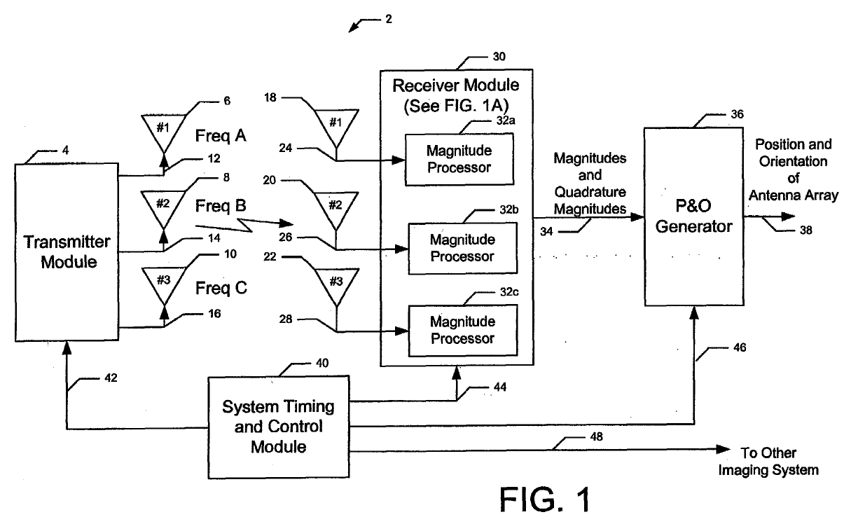

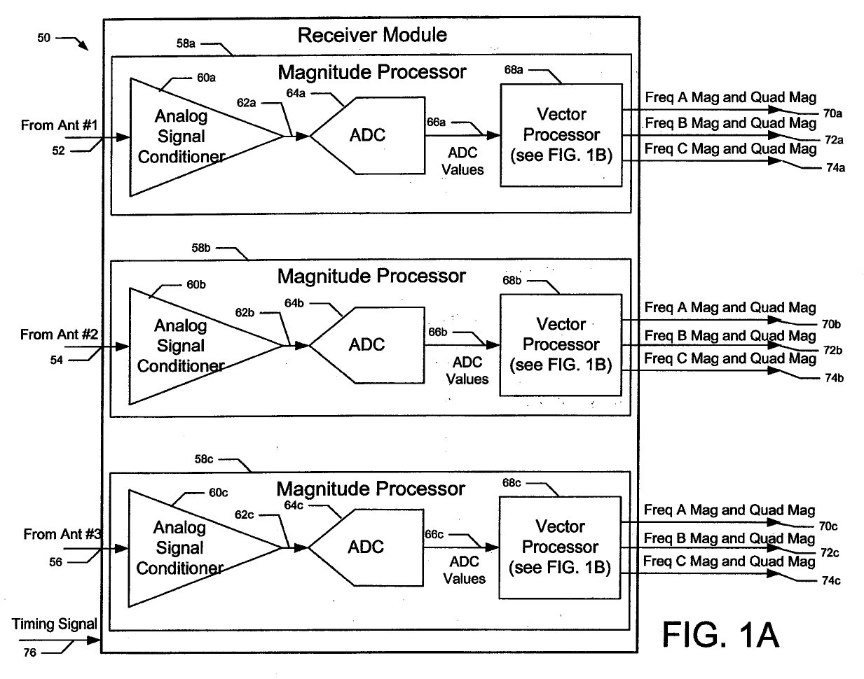

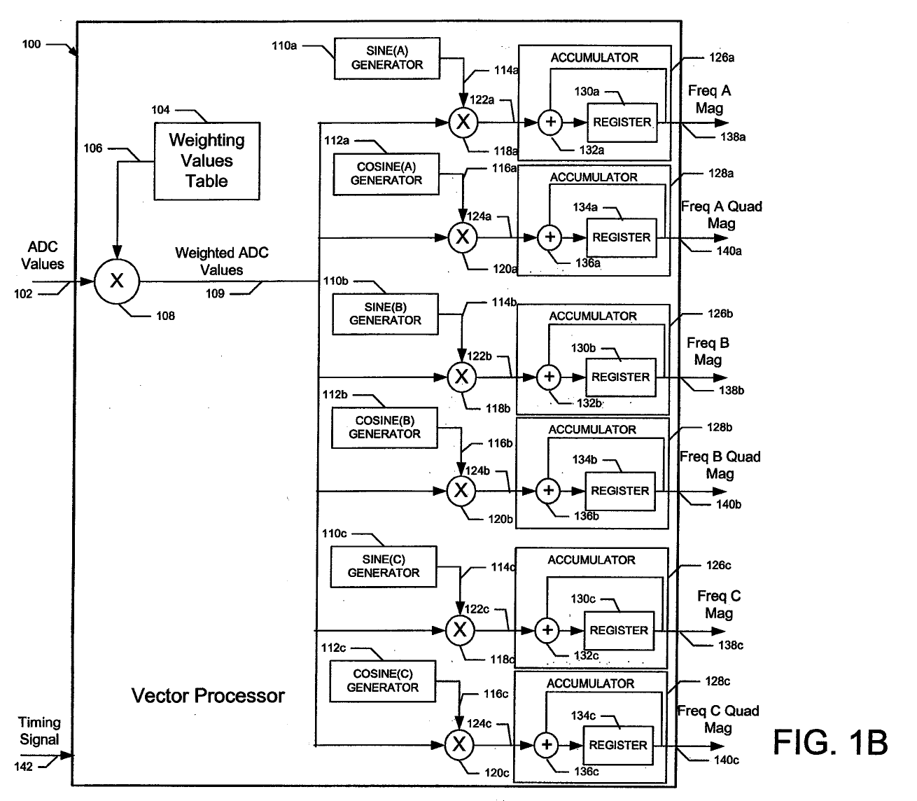

Referring to FIG. 1, an exemplary system 2 includes a plurality of transmitting antennas 6-10, coupled to a transmitter module 4, which is adapted to provide signals 12-16 to the transmitting antennas 6-10, respectively. The transmitter module 4 is coupled to receive a timing signal 42 from a system timing and control module 40.

The system 2 also includes a plurality of receiving antennas 18-22, each coupled to provide a respective signal 24-28 to a respective magnitude processor 32a-32c within a receiver module 30. The receiver module 30 is coupled to receive a timing signal 44 from the system timing and control module 40.

The system 2 also includes a position and orientation (P&O) generator 36 coupled to receive a magnitude signal 34 from the receiver module 30. The P&O generator 36 is coupled to receive a timing signal 46 from the system timing and control module 40.

In operation, the transmitter module 4 communicates the signals 12-16 to the transmitting antennas 6-10. Each one of the signals 12-16 includes at least one narrowband frequency. In one particular embodiment, each one of the signals 12-16 includes a different narrowband frequency. In some embodiments, each one of the signals 12-16 can include more than one narrowband signal. However, in discussion below, it will be assumed that each one of the signals 12-16 includes one different narrowband signal. In some embodiments, the three narrowband signals have different frequencies, each of about 14 kHz. In some embodiments, the three transmitting antennas 6-10 are microcoil antennas.

The transmitting antennas 6-10 convert the signals 12-16 into corresponding electromagnetic signals that propagate to the receiving antennas 18-22. Each one of the receiving antennas 18-22 receives electromagnetic signals in accordance with all three of the signals 12-16. Thus, in some embodiments, each receiving antenna 18-22 receives all three of the electromagnetic signals transmitted by the three transmitting antennas 6-10, each having one narrowband frequency. However, it will be understood that, due in part to positional differences, each one of the receiving antennas 18-22 receives each one of the electromagnetic signals transmitted by the three transmitting antennas 6-10 with a different magnitude and phase. In some embodiments, the three receiving antennas 18-22 are microcoil antennas.

Taking the receiving antenna 18 as representative of the other two receiving antennas 20, 22, the receiving antenna 18 provides a signal 24 having the three narrowband frequencies (each with a particular amplitude and phase) to the magnitude processor 32a. The magnitude processor 32a generates magnitude signals according to each one of the three frequencies received by the antenna 18. In some embodiments, the magnitude processor 32a generates both magnitudes and quadrature magnitudes, as further described below in conjunction with FIGS. 1A and 1B. One of ordinary skill in the art will understand that a magnitude and a quadrature magnitude can be processed together to compute a magnitude and a phase of a signal. In other embodiments, the magnitude processor 32a directly generates a magnitude and a phase of each one of the three frequencies received by the antenna 18.

Similarly, the magnitude processor 32b generates magnitudes and quadrature magnitudes of each one of the three frequencies received by the antenna 20 and the magnitude processor 32c generates magnitudes and quadrature magnitudes of each one of the three frequencies received by the antenna 22. The magnitude processors 32a-32c are described in greater detail below in conjunction with FIGS. 1A and 1B.

All of the magnitudes and quadrature magnitudes, or more simply, magnitudes 34 are communicated to the P&O generator 36, which can compute a position and, in some embodiments, an orientation, of an object being tracked by the system 2, in response to the magnitudes 34. The system is reciprocal, meaning that the three transmitting antennas 6-10 can be coupled to the object being tracked, or alternatively, the three receiving antennas 18-22 can be coupled to the object.

Functions of the P&O generator 36 are not described more fully herein. However, functions of the P&O generator 36 can be as described, for examples, in U.S. patent application Ser. No. 10/611,112, filed Jul. 1, 2003, entitled “Electromagnetic Tracking System Method Using Single-Coil Transmitter,” U.S. Pat. No. 7,015,859, issued Mar. 21, 2006, entitled “Electromagnetic Tracking System and Method Using a Three-Coil Wireless Transmitter,” U.S. Pat. No. 5,377,678, issued Jan. 3, 1995, entitled “Tracking System to Follow the Position and Orientation of a Device with Radiofrequency Fields,” U.S. Pat. No. 5,251,635, issued Oct. 12, 1993, entitled “Stereoscopic X-Ray Fluoroscopy System Using Radiofrequency Fields,” U.S. Pat. No. 6,445,943, issued Sep. 3, 2002, entitled “Position Tracking System for Use in Medical Applications,” U.S. Pat. No. 6,980,921, issued Dec. 27, 2005, entitled “Magnetic Tracking System,” and U.S. Pat. No. 6,774,624, issued Aug. 10, 2004, entitled “Magnetic Tracking System.” Each of the above-identified patent applications and issued patents is incorporated herein by reference in its entirety.

Timing provided by the system timing and control module 40 is described more fully below. Let it suffice, however, to say here that the system timing and control module 40 controls transmissions of the electromagnetic signals by the transmitting antennas 6-10 and associated timing of processing of the electromagnetic signals by the receiver module 30 and P&O generator 36. As a result, the transmissions and the receptions have predetermined gaps therebetween, during which no electromagnetic energy is transmitted or received by the system 2. The gaps can be sufficiently short that the tracking system 2 can still provide good tracking performance with a rapid update rate (frame rate), yet sufficiently long that images from other imaging systems (not shown) can be captured and displayed during one or more of the gaps without electromagnetic interference.

Referring now to FIG. 1A, a receiver module 50 can be the same as or similar to the receiver module 30 of FIG. 1. The receiver module 50 can include three magnitude processors 58a, 58b, 58c. Taking the magnitude processor 58a as representative of the other magnitude processors 58b, 58c, the magnitude processor 58a includes an analog signal conditioner 60a, which can include amplifiers, filters, demultiplexers, or the like. The analog signal conditioner 60a is adapted to receive a signal 52 from a first antenna (e.g., receiving antenna 18 of FIG. 1).

The analog signal conditioner 60a provides an analog signal 62a, having the above-described three narrowband frequencies, to an analog-to-digital converter 64a (ADC). The ADC 64a provides a digitized and time-sampled version 66a (referred to herein as ADC values) of the analog signal 62a to a vector processor 68a. The vector processor 68a is adapted to generate a magnitude and a quadrature magnitude 70a of a first one of the three narrowband frequencies received by the first antenna (e.g., receiving antenna 18 of FIG. 1), a magnitude and a quadrature magnitude 72a of a second one of the three narrowband frequencies received by the first antenna, and a magnitude and a quadrature magnitude 74a of a third one of the three narrowband frequencies received by the first antenna.

Similarly, the magnitude processor 58b includes an analog signal conditioner 60b adapted to receive a signal 54 from a second antenna (e.g., receiving antenna 20 of FIG. 1). The magnitude processor 58b also includes an analog signal conditioner 60b, an ADC 64b, and a vector processor 68b, all coupled as in the magnitude processor 58a. The vector processor 68b is adapted to generate a magnitude and a quadrature magnitude 70b of a first one of the three narrowband frequencies received by the second antenna, a magnitude and a quadrature magnitude 72b of a second one of the three narrowband frequencies received by the second antenna, and a magnitude and a quadrature magnitude 74b of a third one of the three narrowband frequencies received by the second antenna.

Similarly, the third magnitude processor 58c includes an analog signal conditioner 60c adapted to receive a signal 56 from a third antenna (e.g., receiving antenna 22 of FIG. 1). The magnitude processor 58c also includes an analog signal conditioner 60c, an ADC 64c, and a vector processor 68c, all coupled as in the magnitude processor 58a. The vector processor 68c is adapted to generate a magnitude and a quadrature magnitude 70c of a first one of the three narrowband frequencies received by the third antenna, a magnitude and a quadrature magnitude 72c of a second one of the three narrowband frequencies received by the third antenna, and a magnitude and a quadrature magnitude 74c of a third one of the three narrowband frequencies received by the third antenna.

As described above in conjunction with FIG. 1, any magnitude and quadrature magnitude pair can be used to calculate a magnitude and a phase of a signal. For example, the magnitude and quadrature magnitude 70a can be used to calculate a magnitude and a phase of the first one of the three narrowband frequencies received by the first receiving antenna.

Timing of the functions of the receiver module 50 is controlled by a timing signal 76, which can be the same as or similar to the timing signal 44 of FIG. 1.

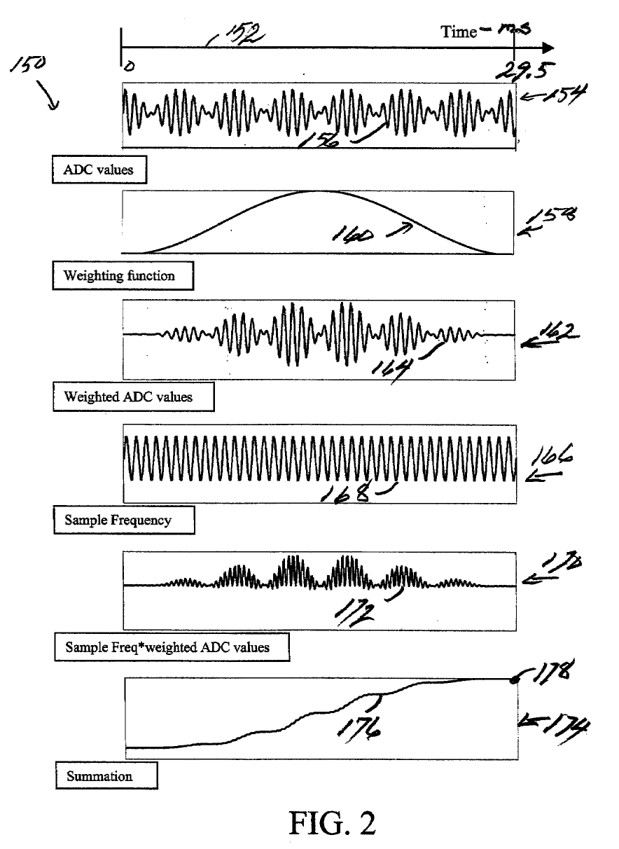

Referring now to FIG. 1B, a vector processor 100 is the same as or similar to one of the vector processors 68a-68c of FIG. 1A. The vector processor 100 includes a multiplier 108, which multiplies ADC values 102 by weighting values 106 stored in a weighting value table 104, resulting in weighted ADC values 109. The ADC values 102 can be the same as or similar to one of the ADC values signals 66a-66c of FIG. 1A. Significance of the weighting values 106 is further described below in conjunction with FIG. 2.

The weighted ADC values 109 are received by a plurality of multipliers 118a-118c and 120a-120c. At the multiplier 118a, the weighted ADC values 109 are multiplied by a sine signal 114a generated by a sine(A) generator 110a, where the designation “A” corresponds to one of the three narrowband frequencies, A, B, C, received by each one of the three antennas 18-22 of FIG. 1. At the multiplier 120a, the weighted ADC values 109 are multiplied by a cosine(A) signal 116a generated by a cosine(A) generator 112a. Signal 122a and quadrature signal 124a result from the multiplications.

At the multiplier 118b, the weighted ADC values 109 are multiplied by a sine signal 114b generated by a sine(B) generator 110b, where the designation “B” corresponds to another one of the three narrowband frequencies, A, B, C, received by each one of the three antennas 18-22 of FIG. 1. At the multiplier 120b, the weighted ADC values 109 are multiplied by a cosine(B) signal 116b generated by a cosine(B) generator 112b. Signal 122b and quadrature signal 124b result from the multiplications.

At the multiplier 118c, the weighted ADC values 109 are multiplied by a sine signal 114c generated by a sine(C) generator 110c, where the designation “C” corresponds to yet another one of the three narrowband frequencies, A, B, C, received by each one of the three antennas 18-22 of FIG. 1. At the multiplier 120c, the weighted ADC values 109 are multiplied by a cosine(C) signal 116c generated by a cosine(C) generator 112c. Signal 122c and quadrature signal 124c result from the multiplications.

In order to generate a magnitude of the signal 122a, a variety of techniques can be used. In one embodiment shown, samples of the signal 122a are accumulated (i.e., added to each other) using an accumulator 126a, which receives the signal 122a at a summing node 132a, and which has a register 130a coupled to the summing node 132a in a feedback arrangement. An output 138a of the register 130a is representative of a magnitude of the signal 122a. Similarly, samples of the signal 124a are accumulated (i.e., added to each other) using an accumulator 128a, which receives the signal 124a at a summing node 136a, and which has a register 134a coupled to the summing node 136a in a feedback arrangement. An output 140a of the register 134a is representative of a magnitude of the quadrature signal 124a. Taken together, it will be understood that the signal magnitude 138a and the quadrature signal magnitude 140a can be used to compute a magnitude and a phase of the narrowband frequency A received by one of the receiving antennas 18-22.

Similarly, accumulators 126b, 128b generate outputs 138b, 140b, which can be used to compute a magnitude and a phase of the narrowband frequency B received by the antenna 18 of FIG. 1. Similarly, accumulators 126c, 128c generate outputs 138c, 140c, which can be used to compute a magnitude and a phase of the narrowband frequency C received by the antenna 18 of FIG. 1.

As described above, having a magnitude and a quadrature magnitude, it should be understood that a magnitude and phase of each one of the narrowband frequencies received by one of the receiving antennas 18-22 can be determined.

In other arrangements, other means can be used to generate magnitude and phase or magnitude and quadrature magnitude of each one of the frequencies A, B, C received by one of the receiving antennas 18-22 of FIG. 1. In yet other arrangements, discrete Fourier transform processors, for example, fast Fourier transform processors, can be used in place of the accumulators 126a-c, 128a-c, multipliers 118a-118c, 120a-120c, sine generators 110a-110c, and cosine generators 112a-112c.

Timing of the functions of the vector processor 100 is controlled by a timing signal 142, which can be the same as or similar to the timing signal 44 of FIG. 1.

The vector processor 100 represents but one of the vector processors 68a-68c of FIG. 1A. Vector processors, the same as or similar to the vector processor 100, can be used within each of the magnitude processors 58a, 58b, 58c of FIG. 1A, to generate signals and quadrature signals for each one of the three narrowband frequencies A, B, C received by each one of the three receiving antennas 18-22 of FIG. 1.

Referring now to FIG. 2, a set of graphs 150 illustrates processing by a vector processor, for example, the vector processor 100 of FIG. 11B. Each graph includes a horizontal scale in units of time in milliseconds. A time scale 152 is representative of the time scales associated with each member of the set of graphs 150. Each graph includes a vertical scale in units of amplitude in arbitrary units. It will be understood that curves shown in each of the graphs are representative of time samples (e.g., digital samples) of signals, graphically connected together for clarity to form continuous curves or signals.

A graph 154 includes a curve 156 (a signal) representative of the signal 102 (ADC values) of FIG. 1B. The curve 156 is comprised of a sum of the above-described three narrowband frequencies, resulting in an amplitude modulation.

A graph 158 includes a curve 160 (a signal) representative of the weighting values 106 of FIG. 1B.

A graph 162 includes a curve 164 (a signal) representative of the weighted ADC values 109 of FIG. 1B, generated by a product of the weighting values 160 and the ADC values 156.

A graph 166 includes a curve 168 (a signal) representative of one of the sine signals 114a-114c of FIG. 1B. The curve 168 has a frequency selected to be one of the narrowband frequencies within the curve 156.

A graph 170 includes a curve 172 (a signal) representative of one of the signals 122a-122c of FIG. 1B generated by a product of the weighted ADC values 164 and the curve 168. It will be understood that the product of the curve 164 with the curve 168 (i.e., the curve 172) has a variety of spectral components in the frequency domain resulting from sum and differences of the frequency of the curve 168 with the three narrowband frequencies of the curve 164. The component of primary interest in the curve 172 is at zero frequency, a DC component. The DC component of the curve 172 is representative of a magnitude of the signal 164, and in particular, a magnitude of a frequency of one of the three narrowband frequencies contained in the signal 164 (i.e., at the frequency of the signal 168). Other sum and difference product components in the signal represented by the curve 172 are generally undesirable, but can be reduced relative to the desired DC component by way of the above-described multiplication by the weighting function 160. However, in other embodiments, a filter, e.g., a digital low pass filter, can first filter the signal represented by the curve 172 in order to reduce the undesired sum and difference product components before the signal is accumulated below.

The weighting function represented by the curve 160 can be one of a variety of conventional or unconventional weighting functions. Conventional weighting functions include, but are not limited to, a uniform weighting function, a flat top weighting function, a Hanning weighting function, a Chebychev weighting function, and a Hamming weighting function, each with particular advantages. Weighting functions are known to be used, in particular, in conjunction with fast Fourier transforms, but have similar advantages when used in the processing represented by the set of graphs 150.

The weighting function represented by the curve 160 can be selected in a variety of ways. In one particular embodiment, as described above, the weighting function is selected to reduce non-DC sum and difference product components from the curve 172.

In one particular embodiment, the weighting function is a Dolph-Chebyshev weighting function, resulting in a low pass filter transfer characteristic having about one hundred forty dB attenuation outside of the passband, and a bandwidth of about 40 Hz.

A graph 174 includes a curve 176 (a signal) representative of one of the signals 138a-138c of FIG. 1B, which is generated by accumulating (adding) the values of the curve 172. The curve 176 has a final value 178, which is representative of an amplitude of the one narrowband frequency within the curve 156, which has a frequency corresponding to the curve 168.

As described above, other techniques can be used to identify a magnitude of one or more of the narrowband frequencies within the curve 156. For example, as described above, in other embodiments, a discrete Fourier transform, such as a fast Fourier transform, can be performed upon one of the curves 156, 164, or 172.

As identified on the time scale 152, in some embodiments, the various curves in the set of graphs 150 can have a time duration of about 29.5 milliseconds, which corresponds to a so-called “collection period.” As used herein, the term “collection period” refers to a time period during which new signal magnitudes are achieved. In some tracking systems, collection periods are repeated without a substantial time gap therebetween.

It will be appreciated that a display frame of a tracking display associated with a tracking system can be updated no faster than the collection period. Therefore, the above-described time duration of about 29.5 milliseconds corresponds to a display frame update rate of about thirty frames per second. This frame rate is generally considered to be fast enough so that a tracking image generated by the tracking system (2 of FIG. 1), which shows a position, and in some systems, an orientation, of surgical instrument being used during medical procedure, can be tracked with sufficient accuracy, particularly while the surgical instrument is being moved.

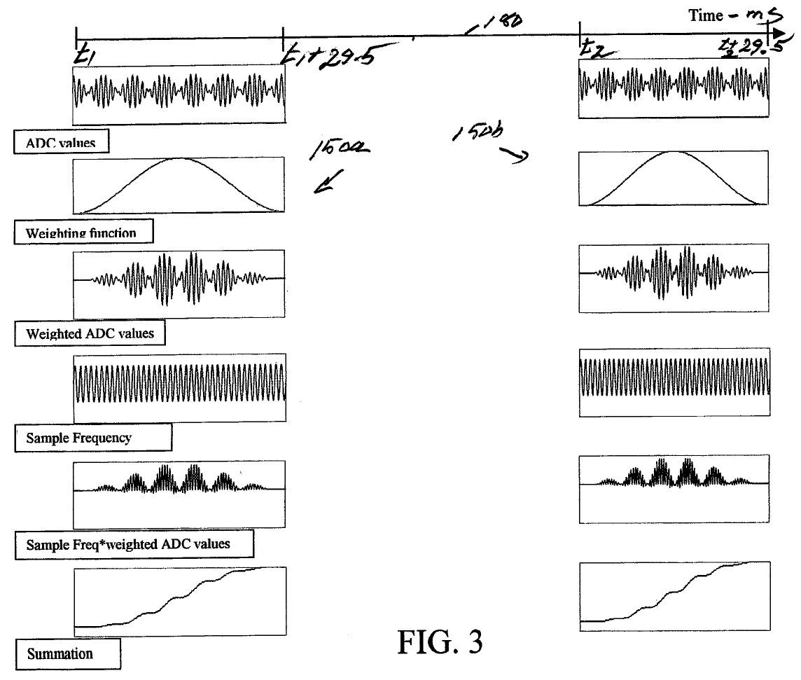

Referring now to FIG. 3, two sets of graphs 150a, 150b, each arranged vertically, are representative of signals described above. Each set of graphs 150a, 150b is substantially the same as the set of graphs 150 of FIG. 2, and therefore, are not described here again in detail.

A time scale 180 represents a time scale of both sets of graphs 150a, 150b and each member of the sets of graphs 150a, 150b has a vertical scale in units of amplitude in arbitrary units. Signals associated with the first set of graphs 150a are collected during a first collection period beginning at a time t1 and ending about 29.5 millisecond later at a time t1+29.5. Signals associated with the second set of graphs 150b are collected during a second collection period beginning at a time t2 and ending about 29.5 millisecond later at a time t2+29.5. During a time gap between times t1+29.5 and t2, no electromagnetic signals are generated or received by the tracking system. During this time gap, other imaging systems are able to capture, process, and display images without electromagnetic interference from the tracking system.

In some arrangements, the time between times t1 and t2 is about sixty-six milliseconds, resulting in a displayed frame rate in the tracking system of about fifteen frames per second, which is relatively slow. In other arrangements, the time between times t1 and t2 is about one hundred thirty five milliseconds, resulting in a displayed frame rate in the tracking system of about 7.5 frames per second, which is unacceptably slow.

By time multiplexing the x-ray imaging system and tracking system data collections, up-date rates (i.e., frame rates) are limited by the sum of T_{RD} and T_{COLL}, where T_{RD} is a time period of an x-ray flat panel detector (FPD) readout, and T_{COLL} is a tracking system collection period. This imposes a limit on tracking system update rate (frame rate). Shortening the collection periods, T_{COLL} (e.g., t1 to t1+29.5) to exactly match a coincidence and length of an x-ray pulse stream would reduce this constraint. However, an ability to discriminate between the three transmitted narrowband frequencies is inversely proportional to the collection period. If the collection period, T_{COLL}, is shortened to be less that about 29.5 milliseconds, a greater frequency spread must be provided between the three narrowband frequencies. The required change in these frequencies can be beyond calibration and characterization range of current electromagnetic tracking systems.

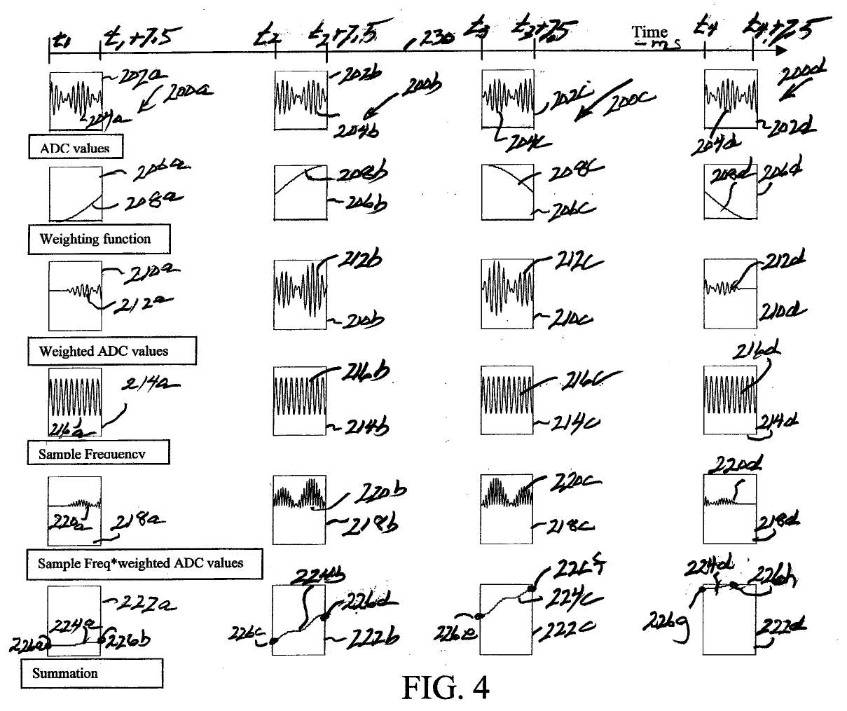

Referring now to FIG. 4, four sets of graphs 200a-200d, each arranged vertically, when taken together, are indicative of signals described above in conjunction with FIG. 2. For example, graphs 202a-202d and associated curves 204a-204d taken together are indicative of the graph 154 and associated curve 156 of FIG. 2. Graphs 206a-206d and associated curves 208a-208d taken together are indicative of the weighting function graph 158 and associated curve 160 of FIG. 2. Graphs 210a-210d and associated curves 212a-212d taken together are indicative of the graph 162 and associated curve 164 of FIG. 2. Graphs 214a-214d and associated curves 216a-216d taken together are indicative of the graph 166 and associated curve 168 of FIG. 2. Graphs 218a-218d and associated curves 220a-220d taken together are indicative of the graph 170 and associated curve 172 of FIG. 2. Graphs 222a-222d and associated curves 224a-224d taken together are indicative of the graph 174 and associated curve 176 of FIG. 2.

A time scale 230 in units of milliseconds, is representative of a time scale of all four sets of graphs 200a-200d and each member of the sets of graphs 200a-200d has a vertical scale in units of amplitude in arbitrary units. Signals associated with the first set of graphs 200a are collected and processed during a first “collection sub-period” beginning at a time t1 and ending about 7.5 millisecond later at a time t1+7.5. As used herein, the term “collection sub-period” is used to describe contiguous processing that in itself does not result in a signal magnitude. The signal magnitude can result from a combination of a plurality of collection sub-periods, as will be understood from discussion below.

Signals associated with the second set of graphs 200b are collected and processed during a second collection sub-period beginning at a time t2 and ending about 7.5 millisecond later at a time t2+7.5. Signals associated with the third set of graphs 200c are collected and processed during a third collection sub-period beginning at a time t3 and ending about 7.5 millisecond later at a time t3+7.5. Signals associated with the fourth set of graphs 200d are collected and processed during a fourth collection sub-period beginning at a time t4 and ending about 7.5 millisecond later at a time t4+7.5.

It will be appreciated that processing associated with the sets of graphs 200a-200d can be performed sequentially at or near the end of respective collection sub-periods. However, as further descried below, a final result is not achieved until processing associated with all four sets of graphs 200a-200d is completed.

During time gaps between times t1+7.5 and t2, t2+7.5 and t3, and also t3+7.5 and t4, no electromagnetic signals are generated or received by the tracking system. During these time gaps, other imaging systems are able to capture, process, and display images without electromagnetic interference from the tracking system.

In some embodiments, times t1, t2, t3, and t4 can be separated by about thirty-three milliseconds, resulting in time gaps of about 25.5 milliseconds. Therefore a total time from time t1 to time t4+7.5 can be about 99+7.5 or 106.5 milliseconds.

It should be apparent that the signals corresponding to the sets of graphs 200a-200d can be collected and processed individually during respective time periods. Once the processing associated with all four sets of graphs 200a-200d is completed, results can be combined to provide the same resulting magnitude value described above in conjunction with FIG. 2. For example, the accumulated summation represented by the curve 224a results in a “partial” summation represented by a point 226b. If the summation represented by the curve 224b begins at a point 226c, having a value substantially equal to the value of the point 226b, then a point 226d is essentially a particle sum of the values of the curves 224a and 224b. If the summation represented by the curve 224c begins at a point 226e, having a value substantially equal to the value of the point 226d, then a point 226f is essentially a partial sum of the values of the curves 224a, 224b, and 224c. If the summation represented by the curve 224d begins at a point 226g, having a value substantially equal to the value of the point 226f, then a point 226h is essentially a “total” sum of the values of the curves 224a, 224b, 224c, and 224d. As described above, it will be understood that the final result represented by the point 226h is achieved only after the processing of all four sets of graphs 200a-220d is completed. The value of the point 226h will be understood to correspond to the value of the point 178 of FIG. 2.

Once the final result represented by the point 226h is achieved, then signals are collected and processed in four more collection sub-periods.

The transmitted signals and associated ADC value signals represented by the curves 204a-204d are substantially synchronous. Therefore, the transmitted signals can be transmitted and received as portions, represented by received ADC value signal portions 204a-204d, and processed in portions, including multiplying the ADC value signal portions 204a-204d individually by portions 208a-208d, respectively, of a weighting function. The portions 208a-208d of the weighting function taken together (i.e., without interposed time gaps) are comparable to the weighting function 160 of FIG. 2.

The sets of graphs 200a-200d are shown to be in a sequence, which, when taken together, without interposing time gaps, has the same curve shapes as the set of graphs 150 of FIG. 2. However, because the transmitted signals associated with the ADC value signals 204a-204d are synchronous, the order of the sets of graphs 200a-200d, and therefore, the order of the transmitted signals associated with the ADC value signals 204a-204d, can be in any order, and processing can be in any order accordingly.

Data collections and processing associated with the sets of graphs 200a-200d is achieved as a group, and the computation is completed once the point 226h is achieved. The processing can be performed on the data of the four collection sub-periods together, i.e., after all data is collected, or in sequence. Thereafter, signals represented by another similar four sets of graphs are collected and processed. A total time for collection and processing extends from time t1 to time t4+7.5.

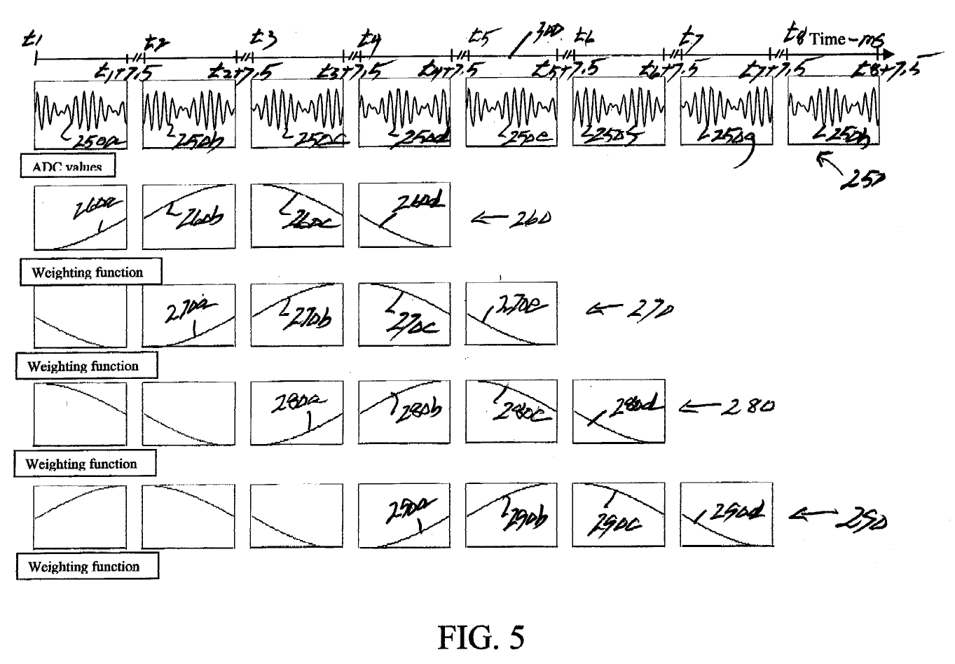

As described above, a total time from time t1 to time t4+7.5 can be about 106.5 milliseconds. If, as described above, the final result (i.e., point 226h) is achieved only when signals associated with all four sets of graphs 200a-200d are collected and processed, the display frame update rate can be as slow as about ten frames per second, which is unacceptably slow. However, as will become apparent from discussion below in conjunction with FIG. 5, the processing associated with the sets of graphs 200a-200d can be sequenced in a different way (e.g., interleaved), resulting in a faster display frame rate.

Referring now to FIG. 5, five sets of graphs 250, 260, 270, 280, 290, are arranged horizontally. Each graph has a horizontal scale in units of time in milliseconds and a vertical scale in arbitrary units of amplitude. A time scale 300 is representative of a time scale for all four sets of graphs 250-290.

The first set of graphs 250 includes curves 250a-250h (signals), which are representative of the signal 102 (ADC values) of FIG. 1B. The signals 250a-250h are comprised of three narrowband frequencies, resulting in an amplitude modulation.

A signal 250a is collected during a first collection period (as opposed to a collection sub-period for reasons described more fully below) beginning at a time t1 and ending about 7.5 millisecond later at a time t1+7.5. As described above, the term collection period is used to describe a period in which data are collected and a final magnitude result is achieved. A signal associated with a curve 250b is collected during a second collection period beginning at a time t2 and ending about 7.5 millisecond later at a time t2+7.5. A signal 250c is collected during a third collection period beginning at a time t3 and ending about 7.5 millisecond later at a time t3+7.5. A signal 250d is collected during a fourth collection period beginning at a time t4 and ending about 7.5 millisecond later at a time t4+7.5. A signal 250e is collected during a fifth collection period beginning at a time t5 and ending about 7.5 millisecond later at a time t5+7.5. A signal 250f is collected during a sixth collection period beginning at a time t6 and ending about 7.5 millisecond later at a time t6+7.5. A signal 250g is collected during a seventh collection period beginning at a time t7 and ending about 7.5 millisecond later at a time t7+7.5. A signal 250h is collected during an eighth collection period beginning at a time t8 and ending about 7.5 millisecond later at a time t8+7.5.

The signals 250a-250h are separated in time from adjacent signals by time gaps between times t1+7.5 and t2, t2+7.5 and t3, t3+7.5 and t4, t4+7.5 and t5, t5+7.5 and t6, t6+7.5 and t7, and also t7+7.5 and t8, respectively. In some embodiments, the times t1, t2, t3, t4, t5, t6, t7, and t8 are each separated by about thirty-three milliseconds, resulting in time gaps of about 25.5 milliseconds.

The set of graphs 260 includes curves 260a-260d (signals), which are each individually representative of a respective portion of the above-described weighting function (e.g., 160, FIG. 2). The curves 260a-260d can be the same as or similar to the curves 208a-208d of FIG. 4.

The set of graphs 270 includes curves 270a-270d (signals), which are each individually representative of a respective portion of the above-described weighting function. The curves 270a-270d can be the same as or similar to the curves 208a-208d of FIG. 4, but delayed in time by one collection period from the curves 260a-260d.

The set of graphs 280 includes curves 280a-280d (signals), which are each individually representative of a respective portion of the above-described weighting function. The curves 280a-280d can be the same as or similar to the curves 208a-208d of FIG. 4, but delayed in time by one collection period from the curves 270a-270d.

The set of graphs 290 includes curves 290a-290d, which are each individually representative of a respective portion of the above-described weighting function. The curves 290a-290d can be the same as or similar to the curves 208a-208d of FIG. 4, but delayed in time by one collection period from the curves 280a-280d.

The four sets of graphs are offset from each other in time by one collection period. The ADC value signals 250a-250d are collected sequentially during collection periods t1 to t1+7.5, t2 to t2+7.5, t3 to t3+7.5, and t4 to t4+7.5 respectively. The signals 250a-250d can be processed sequentially, essentially as described above in conjunction with FIG. 4, or they can be processed as a group at or near the end time t4+7.5 (i.e., at substantially the same time). In either arrangement, as described above in conjunction with FIG. 4, a final result is achieved only when all four signals 250a-250d are collected and processed, i.e., after time t4+7.5. The processing of the four signals 250a-250d with the signals 260a-260d, whether sequentially processed or processed at substantially the same time, constitute a full collection period.

Processing associated with the curves 260a-260d can be performed in a collection period in much the same way as the processing discussed in conjunction with FIG. 4, resulting in signals comparable to the other signals of FIG. 4, including the accumulated signals 224a-224d. The collection period associated with signals 260a-260d employs the signals 250a-250d, and is completed at or near the time t4+7.5, at which time a first final magnitude result is achieved (e.g., the point 226h of FIG. 4)

Processing associated with the curves 270a-270d can also be performed in a collection period in much the same way as the processing discussed in conjunction with FIG. 4, resulting in signals comparable to the other signals of FIG. 4, including the accumulated signals 224a-224d. The collection period associated with signals 270a-270d employs the signals 250b-250e, and is completed at or near the time t5+7.5, at which time a second final magnitude result is achieved, comparable to another point 226h of FIG. 4.

In one particular embodiment, in accordance with time delays between t1, t2, t3, t4, t5, t6, t7, and t8 of thirty three milliseconds, the processing associated with the signals 270a-270d is completed about thirty three milliseconds after the above-described processing associated with the signals 260a-260d. For this reason, the term collection period (rather than collection sub-period) is used to describe the periods t1 to t1+7.5, t2 to t2+7.5, etc.

In substantially the same way, a collection period associated with signals 280a-280d employs the signals 250c-250f, and is completed at or near the time t6+7.5, at which time a third final magnitude result is achieved, comparable to another point 226h of FIG. 4. Similarly, a collection period associated with signals 290a-290d employs the signals 250d-250g, and is completed at or near the time t7+7.5, at which time a fourth final magnitude result is achieved, comparable to another point 226h of FIG. 4. It will be appreciated that the collection periods are interleaved, each collection period using some of the same signals 250a-250g.

Unlike the processing described in conjunction with FIG. 4, which, as described above, achieves a display frame rate only of about ten frames per second, the interleaved processing described in conjunction with FIG. 5 achieves a frame rate of about thirty frames per second, which is reasonably fast.

While systems and techniques are described above which have three transmitting antennas, three receiving antennas, and three narrowband frequencies, in other embodiments, more than three or fewer than three transmitting antennas, more than three or fewer than three receiving antennas, and more than three or fewer than three narrowband frequencies can be used. Furthermore, the number of transmitting antennas, the number of receiving antennas, and the number of narrowband frequencies need not match.

While particular collection periods and collection sub-periods having particular time durations and time gaps are described above, it will be appreciated that other time durations and other time gaps can be used.

All references cited herein are hereby incorporated herein by reference in their entirety.

Having described preferred embodiments of the invention, it will now become apparent to one of ordinary skill in the art that other embodiments incorporating their concepts may be used. It is felt therefore that these embodiments should not be limited to disclosed embodiments, but rather should be limited only by the spirit and scope of the appended claims.

What is claimed is:

1. A method of processing a signal to track a surgical instrument, comprising:
transmitting, in a plurality of time windows separated from each other in time, a respective plurality of electromagnetic signals having a respective plurality of time distributions, the plurality of electromagnetic signals including a first electromagnetic signal and a second electromagnetic signal, wherein the first electromagnetic signal is transmitted during a first time window of the plurality of time windows, wherein the first electromagnetic signal has one or more narrowband frequencies, wherein the first electromagnetic signal has a first time distribution in the first time window, wherein the second electromagnetic signal is transmitted during a second time window of the plurality of time windows, wherein the second electromagnetic signal has the one or more narrowband frequencies, wherein the second electromagnetic signal has a second time distribution in the second time window, wherein the first and second time windows are each shorter than a collection period, wherein the second time window is separated in time by a time gap from the first time window, and wherein the time gap is sufficiently long to allow an image to be generated during the time gap;
receiving the first electromagnetic signal during the first time window;
receiving the second electromagnetic signal during the second time window;
processing the first electromagnetic signal together with at least the second electromagnetic signal to provide a magnitude of the one or more narrowband frequencies; and
processing the magnitude of the one or more narrowband frequencies in order to track a position of the surgical instrument.

2. The method of claim 1, wherein a set of electromagnetic signals selected from among the plurality of electromagnetic signals and including the first and second electromagnetic signals has respective time distributions in respective time windows that are continuous.

3. The method of claim 1, wherein the second time distribution of the second electromagnetic signal in the second time window is different than the first time distribution of the first electromagnetic signal in the first time window.

4. The method of claim 3, wherein the second time distribution of the second electromagnetic signal in the second time window is continuous with the first time distribution of the first electromagnetic signal in the first time window.

5. The method of claim 1, wherein the processing the first electromagnetic signal together with at least the second electromagnetic signal comprises:
converting the first electromagnetic signal to a first electronic signal; and
multiplying the first electronic signal by a first portion of a weighting function to provide a first weighted signal;
converting the second electromagnetic signal to a second electronic signal; and
multiplying the second electronic signal by a second portion of the weighting function to provide a second weighted signal, wherein the second portion of the weighting function is different than the first portion of the weighting function.

6. The method of claim 5, wherein the second portion of the weighting function is continuous with the first portion of the weighting function.

7. The method of claim 5, wherein the processing the first electromagnetic signal together with at least the second electromagnetic signal further comprises:
multiplying the first weighted signal by a frequency signal having a selected one of the one or more frequencies to provide a first product signal; and
multiplying the second weighted signal by the frequency signal having the selected one of the one or more frequencies to provide a second product signal.

8. The method of claim 5, wherein the first weighted signal and the second weighted signal are associated with a first collection period interleaved with another collection period.

9. The method of claim 5, wherein the plurality of electromagnetic signals further includes a third electromagnetic signal, wherein the third electromagnetic signal is transmitted during a third time window of the plurality of time windows, wherein the third electromagnetic signal has the one or more narrowband frequencies, wherein the third electromagnetic signal has a third time distribution in the third time window, wherein the third time distribution, the second time distribution, and the first time distribution are different time distributions, and wherein the third time window is separated in time by a second time gap from the second time window, the method further comprising:
receiving the third electromagnetic signal during the third time window; and
processing the first electromagnetic signal and the second electromagnetic signal together with at least the third electromagnetic signal, wherein the processing the first electromagnetic signal and the second electromagnetic signal together with at least the third electromagnetic signal comprises:
converting the third electromagnetic signal to a third electronic signal;
multiplying the second electronic signal by the first portion of the weighting function to provide a third weighted signal; and
multiplying the third electronic signal by the second portion of the weighting function to provide a fourth weighted signal.

10. The method of claim 9, wherein the first weighted signal and the second weighted signal are associated with a first collection period, and wherein the third weighted signal and the fourth weighted signal are associated with a second collection period interleaved with the first collection period.

11. Apparatus for processing a signal to track a surgical instrument, comprising:
a transmitter adapted to transmit, in a plurality of time windows separated from each other in time, a respective plurality of electromagnetic signals having a respective plurality of time distributions, the plurality of electromagnetic signals including a first electromagnetic signal and a second electromagnetic signal, wherein the first electromagnetic signal is transmitted during a first time window of the plurality of time windows, wherein the first electromagnetic signal has one or more narrowband frequencies, wherein the first electromagnetic signal has a first time distribution in the first time window, wherein the second electromagnetic signal is transmitted during a second time window of the plurality of time windows, wherein the second electromagnetic signal has the one or more narrowband frequencies, wherein the second electromagnetic signal has a second time distribution in the second time window, wherein the first and second time windows are each shorter than a collection period, wherein the second time window is separated in time by a time gap from the first time window, and wherein the time gap is sufficiently long to allow an image to be generated during the time gap;
a receiver adapted to receive the first electromagnetic signal during the first time window and the second electromagnetic signal during the second time window;
at least one magnitude processor adapted to process the first electromagnetic signal together with at least the second electromagnetic signal to provide a magnitude of the one or more narrowband frequencies; and
a position and orientation generator adapted to process the magnitude of the one or more narrowband frequencies in order to track a position of the surgical instrument.

12. The apparatus of claim 11, wherein a set of electromagnetic signals selected from among the plurality of electromagnetic signals and including the first and second electromagnetic signals has respective time distributions in respective time windows that are continuous.

13. The apparatus of claim 12, wherein the second time distribution of the second electromagnetic signal in the second time window is different than the first time distribution of the first electromagnetic signal in the first time window.

14. The apparatus of claim 13, wherein the second time distribution of the second electromagnetic signal in the second time window is continuous with the first time distribution of the first electromagnetic signal in the first time window.

15. The apparatus of claim 15, wherein the at least one magnitude processor comprises:
at least one converter adapted to convert the first electromagnetic signal to a first electronic signal and adapted to convert the second electromagnetic signal to a second electronic signal; and
at least one multiplier adapted to multiply the first electronic signal by a first portion of a weighting function to provide a first weighted signal, and adapted to multiply the second electronic signal by a second portion of the weighting function to provide a second weighted signal, wherein the second portion of the weighting function is different than, but continuous with, the first portion of the weighting function.

16. The apparatus of claim 15, wherein the second portion of the weighting function is continuous with the first portion of the weighting function.

17. The apparatus of claim 15 wherein the at least one magnitude processor further comprises:
a second at least one multiplier adapted to multiply the first weighted signal by a frequency signal having a selected one of the one or more frequencies to provide a first product signal, and further adapted to multiply the second weighted signal by the frequency signal having the selected one of the one or more frequencies to provide a second product signal.

18. The apparatus of claim 15, wherein the first weighted signal and the second weighted signal are associated with a first collection period interleaved with another collection period.

19. The apparatus of claim 15, wherein the plurality of electromagnetic signals further includes a third electromagnetic signal, wherein the third electromagnetic signal is transmitted during a third time window of the plurality of time windows, wherein the third electromagnetic signal has the one or more narrowband frequencies, wherein the third electromagnetic signal has a third time distribution in the third time window, wherein the third time distribution, the second time distribution, and the first time distribution are different time distributions, wherein the third time window is separated in time by a second time gap from the second time window, wherein the receiver is further adapted to receive the third electromagnetic signal during the third time window, wherein the at least one converter is further adapted to convert the third electromagnetic signal to a third electronic signal, wherein the at least one multiplier is further adapted to multiply the second electronic signal by the first portion of the weighting function to provide a third weighted signal and to multiply the third electronic signal by the second portion of the weighting function to provide a fourth weighted signal.

20. The apparatus of claim 19, wherein the first weighted signal and the second weighted signal are associated with a first collection period, and wherein the third weighted signal and the fourth weighted signal are associated with a second collection period interleaved with the first collection period.

21. A method of processing a signal to track a surgical instrument, comprising:
collecting and processing electromagnetic signals in first and second collection periods, wherein the first and second collection periods are interleaved.

22. The method of claim 21, further including:
collecting and processing each one of the electromagnetic signals in a collection sub-period associated with both the first and second collection periods.

23. Apparatus for processing a signal to track a surgical instrument, comprising:
a processor adapted to collect and to process electromagnetic signals in first and second collection periods, wherein the first and second collection periods are interleaved.

24. The apparatus of claim 23, wherein the processor is further adapted to collect and to process each one of the electromagnetic signals in a collection sub-period associated with both the first and second collection periods.
